(19) United States
(12) Patent Application Publication
(10) Pub. No.: US 2014/0350669 A1
(43) Pub. Date: Nov. 27, 2014

(54) PERCUTANEOUS VALVE REPLACEMENT DEVICES

(71) Applicant: The Trustees of The University of Pennsylvania, Philadelphia, PA (US)

(72) Inventors: Matthew J. Gillespie, Bryn Mawr, PA (US); Joseph H. Gorman, Gwynedd, PA (US); Robert C. Gorman, Lower Gwynedd, PA (US)

(73) Assignee: The Trustees if The University of Pennsylvania, Philadelphia, PA (US)

(21) Appl. No.: 14/361,466

(22) PCT Filed: Nov. 30, 2012
(86) PCT No.: PCT/US12/67339

§ 371 Date: May 29, 2014

Related U.S. Application Data
(60) Provisional application No. 61/565,958, filed on Dec. 1, 2011.

(51) Int. Cl. A61F 2/24

(52) U.S. Cl. CPC A61F 2/2418; A61F 2/2442; A61F 2250/006
USPC 623/2.18

(57) ABSTRACT

A self-expanding valved stent is constructed from a polytetrafluoroethylene (PTFE) covered nitinol or stainless steel wire frame. Anchoring is facilitated by arms emanating from the ventricular end of the device that are designed to atraumatically insinuate themselves around chordae and leaflets and trap them against the expanded stent body. The valve prosthesis includes a partially self-expanding stent having a wire framework defining outer and interior surfaces and anchoring arms. The stent has an unexpaneled and an expanded state and anchoring arms having an elbow region and a hook that clamps around mitral tissue of the patient when seated. An elastic fabric/cloth made of for example, PTFE material, is wrapped circumferentially around the wire framework. A valve having at least one leaflet is fixedly attached to the interior surface of the stent.

### CROSS-REFERENCE TO RELATED APPLICATIONS

The present patent application claims priority to U.S. Provisional Patent Application No. 61/565,958 filed Dec. 1, 2011. The content of that patent application is hereby incorporated by reference in its entirety.

### TECHNICAL FIELD

The present invention relates to percutaneous valve replacement devices and, in particular, to percutaneous valve replacement devices that provide optimal anchoring and sealing when the device is seated within the cone-shaped space created by the annulus and leaflets.

### BACKGROUND

The mitral valve is a complex structure whose competence relies on the precise interaction of annulus, leaflets, chordae, papillary muscles and the left ventricle (LV). Pathologic changes in any of these structures can lead to mitral regurgitation (MR). Ischemic mitral regurgitation (IMR) occurs when a structurally normal mitral valve (MV) is rendered incompetent as a result of LV remodelling induced by myocardial infarction (MI).

IMR affects 2.4 million Americans and is present in some degree in over 50% of patients with reduced LV ejection fraction undergoing coronary artery bypass grafting (CABG). The magnitude of this clinical problem is significant and expected to grow substantially as the population ages. IMR increases mortality even when mild, with a strongly graded relationship between severity and reduced survival. Currently, IMR can be treated with either mitral valve repair or replacement. Mitral valve repair with undersized ring annuloplasty, typically performed in conjunction with CABG, has become the preferred treatment. However, this therapeutic approach is associated with a 30% recurrence rate of IMR at 6 months after surgery with recurrence approaching 60% at 3 to 5 years. This lack of durability has likely contributed to the difficulty in demonstrating a survival advantage of MV repair compared with either medical management, or with revascularization alone. These reports have generated much discussion in the cardiac surgery world regarding repair versus replacement in the treatment of IMR.

Regardless of the surgical debate, it should be understood that the vast majority of patients with moderate to severe IMR and associated congestive heart failure (CHF) are never treated surgically. It is estimated that less than 2% of the 2.4 million IMR patients in the US receive surgical correction. IMR can intermittently and unexpectedly destabilize the heart failure patient requiring increased medication and repeated hospitalizations. While it is still unclear from scientific investigation whether restoring mitral valve function in these patients will improve survival, there is general consensus that it would make the care of many of them more effective and less costly. Despite this understanding, the risk of surgery for these patients is deemed prohibitive because of the need for a relatively large incision and the morbidity of cardiopulmonary bypass (CPB).

This large unmet clinical need drove the development of several transcatheter mitral valve repair techniques during the early part of the 2000s. Despite early optimism, a number of issues have proven problematic with all these devices including inability to demonstrate effective proof of concept and clinical efficacy. The major reason for these failures is likely due to the fact that all transcatheter repair techniques are only partial approximation of open surgical repair which in itself has been shown to be less efficacious than thought only a decade ago.

In contrast to the failure of catheter based valve repair techniques, catheter based heart valve replacement technology has been successful enough to produce the initiation of a major paradigm shift in valve therapy. Improvements in imaging, catheter technology, and stent design have combined to make transcatheter replacement of the aortic and pulmonic valves clinical realities. These valves can be placed via a peripheral blood vessel or by a tiny thoracotomy without the need for CPB. These successes combined with the growing understanding of the inadequacies of mitral valve repair have piqued interest in the development of transcatheter mitral valve replacement technologies.

Three groups have published the results of their attempts to develop a feasible approach to TMVR in animal models. All have reported limited success and identified similar difficulties. The first obstacle is the lack of adequate echocardiographic visualization or fluoroscopic landmarks of the mitral valve apparatus for device deployment. The second barrier is related to the left ventricular out flow (LVOT) obstruction which results from the exclusive use of radial force to anchor a valved stent inside the mitral annulus. The next two impediments to success are related to the anatomy of the mitral valve apparatus. The complex annular and leaflet geometry makes perivalvular seal a significant challenge while the presence of chordae tendineae can interfere with complete expansion, accurate positioning, and anchorage. The fifth challenge is that the mitral valve must anchor and seal against the highest pressures in the circulation. Thus, the complex anatomy of the mitral valve and the high pressures it is exposed to have prevented the application of the current aortic and pulmonic replacement technologies to the treatment of mitral valve disease.

A transcatheter approach to mitral valve replacement (TMVR) would represent a major advance in the treatment of valvular heart disease since approximately 2.4 million Americans suffer from moderate to severe ischemic mitral regurgitation (IMR) with the vast majority being deemed too sick or debilitated to tolerate open-heart surgery. Successful TMVR requires (1) a sutureless anchoring mechanism, (2) a perivalvular sealing strategy, and (3) foldability. In PCT Application No. PCT/US2010/055645 filed Nov. 5, 2010, the present inventors demonstrated a successful TMVR design that can anchor and seal robustly in large animal models. It is desired in accordance with the present invention to optimize the design of such a TMVR device to maximize device foldability and delivery without compromising valve fixation and seal. The goal of the invention is thus to further hone the design of the TMVR device to increase the device's flexibility which will facilitate transcatheter deliverability and enhance perivalvular seal while maintaining anchoring strength. Such a TMVR device is believed to have the potential to provide an improved treatment strategy for hundreds of thousands of patients annually.

### SUMMARY

The present inventors have addressed the above needs in the art by developing an improved anchoring and sealing mechanism for TMVR. The exemplary embodiments include a self-expanding valved stent constructed from a polytetrafluoroethylene (PTFE) covered nitinol wire frame. Anchoring is facilitated by arms emanating from the ventricular end of the device which are designed to atraumatically insinuate themselves around chordae and leaflets. The sealing mechanism relies on the flexibility of the stent, which allows the device to be slightly oversized, thereby permitting it to conform snuggly to the annulus and leaflet cone.

The valve prosthesis of the invention is described by way of exemplary embodiments with and without an annuloplasty ring. In a first embodiment, the valve prosthesis includes an at least partially self-expanding stent comprising a wire framework defining outer and interior surfaces and an anchoring arm. The stent has an unexpanded and an expanded state. The anchoring arm has an elbow region and a hook that clamps around mitral tissue of the patient when seated. An elastic fabric/cloth made of, for example, PTFE material, is wrapped circumferentially around the wire framework. The wire framework itself traverses the circumference of the stent with a pitch may extend a portion of the length of the stent or may extend the entire length of the stent 4-10 times. A valve comprising at least one leaflet is fixedly attached to the interior surface of the stent. In exemplary embodiments, the number of anchoring arms is minimized and preferably the stent has no more than 12 anchoring arms. The length of the anchoring arms is also minimized and preferably the anchoring arms have lengths that are 40% of the length of the stent. The anchoring arms may alternatively flare circumferentially outward.

In a second embodiment, a failed mitral valve repair is treated using an annuloplasty ring. This embodiment makes stent replacement of the valve much easier and the anchoring arms are not needed to anchor the valve prosthesis. In this embodiment, the valve prosthesis includes an at least partially self-expanding stent comprising a wire framework defining outer and interior surfaces and the stent has an unexpanded and an expanded state. However, the anchoring arms are optional in this embodiment. An elastic fabric/cloth made of, for example, PTFE material, is wrapped circumferentially around the wire framework and a valve having at least one leaflet is fixedly attached to the interior surface of the stent. However, in this embodiment, an annuloplasty ring is provided into which the stent is inserted prior to expansion. The stent is adapted to be expanded to be held in place by radial pressure against the annuloplasty ring. The annuloplasty ring and/or the stent also may have a magnet and/or a detent incorporated therein such that the expanded stent does not move relative to the annuloplasty ring.

### BRIEF DESCRIPTION OF THE DRAWINGS

The various novel aspects of the invention will be apparent from the following detailed description of the invention taken in conjunction with the accompanying drawings, of which:

FIG. 1 illustrates images of a prior art mitral valve design of the inventors, where (A) and (B) represent different views of the 0.012 inch nitinol wire weave anchoring and sealing design with a bovine pericardial trileaflet valve in place. (C) illustrates an atrial view of the device after it had functioned effectively in a sheep for one week, and (D) illustrates the same device from a ventricular view.

FIG. 2 illustrates a TMVR device fitting snuggly within the leaflet cone formed by the annulus, anterior leaflet, posterior leaflet, and chordae, which is the position where the device optimally anchors and seals.

FIG. 3 illustrates how the anchoring mechanism of the TMVR device is facilitated by ventricular contraction. (A) illustrates that the device is placed so that the arms are slightly below the leaflets which are held in their open position by the stent, while (B) illustrates that when the device is released the contraction of the left ventricle loads the valve pushing the anchoring arms up behind the leaflets and captures them atraumatically against the stent.

FIG. 4 illustrates a first embodiment of a PTFE-nitinol wire valve prosthetic device in accordance with the invention. The device is shown on the left in expanded position and on the right in its folded transcatheter delivery position.

FIGS. 5A-5C illustrate an embodiment of the device of FIG. 4 where (A) is a side view, (B) is a view of the device from ventricular to atrial end, and (C) is a close up view of the anchoring arm design.

FIGS. 5D-5E show side and end face views of an alternative embodiment of the device of FIGS. 5A-5C in which the wire framework is different than that shown in 5A and 5B.

FIG. 5F shows a radiographic view of the device pictured in 5D-5E implanted within the mitral annulus.

FIG. 5G shows yet another embodiment of the device in which the atrial aspect of the device is flared outward from the center, terminating in atrial arms that enhance device deliverability, anchoring, and seal.

FIG. 6 illustrates at (A)-(F) the mini thoracotomy procedure used for placement of the minimally invasive off-pump mitral valve replacement device of the invention.

FIG. 7 illustrates at (A) and (B) the 3 cm incision surgeons use to repair the mitral valve using CPB and thoracoscopic instruments or robotic surgical techniques.

FIG. 8A illustrates a first exemplary embodiment of a delivery system for delivering the device of FIGS. 4 and 5 to the heart.

FIGS. 8B-8D illustrate in various states of expansion an alternative delivery system in which the peaks of the device frame at the atrial (proximal) end of the device are grabbed by a claw mechanism that collapses the device centrally to reduce the profile for delivery via catheter.

FIGS. 8E-8G illustrate schematic representations of the stepwise expansion and eventual release of the device of FIG. 5G from the claw mechanism of the embodiment of FIGS. 8B-8D.

FIG. 9 illustrates another embodiment of the invention in which a transvenous/transatrial septal approach is used for valved stent-in-Ring (VIR) delivery. In (A) the valved stent device is crimped on the delivery balloon and advanced over the guide wire from the femoral vein, across the atrial septum and positioned centrally in the annuloplasty ring. (B) shows deployment of the valved stent via balloon inflation, while (C) shows a follow-up left ventriculogram. There is no mitral regurgitation and no left ventricular outflow tract obstruction. An atrial closure device is used to close the small atrial septal defect.

### DETAILED DESCRIPTION OF ILLUSTRATIVE EMBODIMENTS

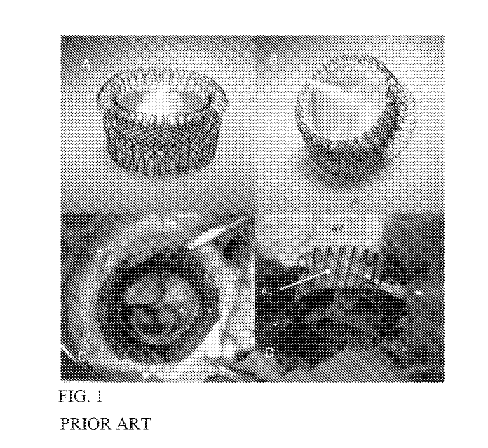

The invention will be described in detail below with reference to FIGS. 1-9. Those skilled in the art will appreciate that the description given herein with respect to those figures is for exemplary purposes only and is not intended in any way to limit the scope of the invention. All questions regarding the scope of the invention may be resolved by referring to the appended claims.

### Overview

The inventors have found that optimal anchoring and seal occurs when the mitral valve replacement device is seated completely within the cone-shaped space created by the annulus and leaflets. Positioning within the leaflet cone is influenced by arm length of the anchoring arms that function to gather tissue centrally to the body of the stent device so as to aid in anchoring and sealing in the mitral opening. If the anchoring arms are too long, the device can be held partially beneath the leaflets causing left ventricular outflow tract (LVOT) obstruction and an ineffective seal. On the other hand, if the anchoring arms are too short, anchoring strength is diminished. The optimal length and number of anchoring arms necessary to anchor and seal the device are described herein. Different designs for use with and without an annuloplasty ring are described.

To determine the optimal number of anchoring arms, prototypes were constructed with four different numbers of arms (20, 16, 12 and 8). Anchoring arm length was kept the same in each (0.75 arm length to stent length ratio—ASR). A pericardial valve was fitted to each and the device was inserted into sheep (80 kg). Because anchoring arm design influences the design of the delivery system, standard cardiac surgical techniques were used. After placement, the valve seal was assessed echocardiographically for stability and perivalvular seal. If function was satisfactory, the valve was reassessed after a month. The design with the fewest number of anchoring arms was further constructed with varying arm lengths (0.6, 0.4, 0.3, 0.2 ASR) and tested in animals. In the testing paradigm, each arm number was tested in 5 animals.

Embodiments of two types of steerable, coaxial, delivery, deployment and retrieval systems will be described below. The first system is designed to allow placement of the valve through a small thoracotomy and atrial purse string. The second system allows for valve placement via a transfemoral vein/transatrial septum approach to the mitral valve. Both systems are tailored to accommodate the determined optimized anchoring arm design of the TMVR device. For each system, the length, width, radius of curvature, release mechanism, and docking station characteristics are defined.

A mini thoracotomy delivery is used and the folding technology is honed to permit percutaneous device placement in a beating heart with or without the use of percutaneous placement catheters. Once placement was achieved reproducibly, a TMVR in accordance with the invention was placed in 5 animals, and the animals were reevaluated by echocardiography after about one month. A transfemoral vein delivery device may also be used.

### Novel Anchoring and Seal Technology

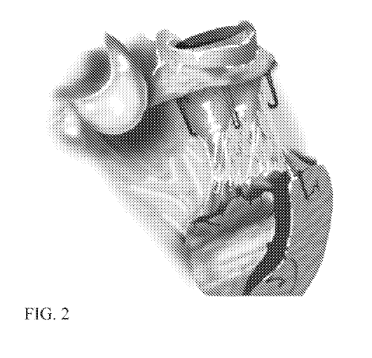

The present invention is directed to a mitral valve prosthesis with a design that overcomes many of the obstacles noted in the background section above. For example, the present inventors have developed the design illustrated in FIG. 1 and described in PCT Application No. PCT/US2010/055645 filed Nov. 5, 2010, the contents of which are incorporated herein by reference. The valve prosthesis described therein uses a 0.012 inch nitinol wire weave design to produce a very flexible stent. The flexibility of the stent allows it to be mildly oversized (2-3 mm greater than the mitral intercommissural diameter), which allows the device to gently conform to the complex mitral annular geometry creating a perivalvular seal without impinging upon the LVOT. As will be appreciated from FIG. 1, the ventricular anchoring arms have insinuated themselves around the anterior leaflet (AL) and the chordae. Additionally, it is evident that the arms do not impinge upon the aortic valve (AV) and have caused no trauma to the heart. The device shown in (C) and (D) of FIG. 1 was placed using standard open heart surgical techniques and represents an effective sutureless mitral valve replacement. The cross clamp time necessary to place this particular device was 8 minutes. The inventors have found the optimal anchoring, seal and avoidance of LVOT impingement occurs when this device is sized (length and diameter) to remain within and conform snuggly to the annulus and leaflet cone as illustrated in FIG. 2.

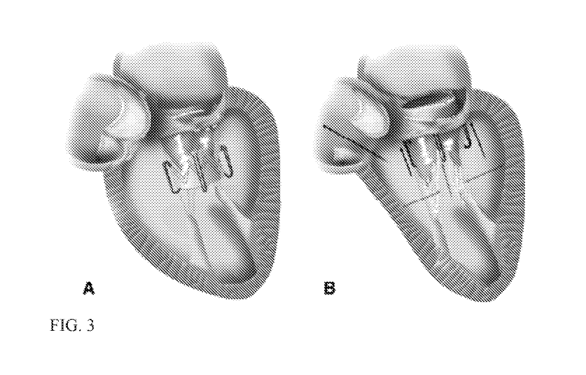

The device of FIG. 1 does not rely on radial force alone for anchoring strength. Anchoring is facilitated by grasping arms which emanate from the ventricular aspect of the stent. These arms have been designed to insinuate themselves around the leaflets and chordae when the device is exposed to systolic LV pressures. This design actually harnesses the LV pressure to help seat the valve in the correct anchoring position as shown in FIG. 3. In particular, FIG. 3 illustrates how the anchoring mechanism of the TMVR device is facilitated by ventricular contraction. (A) illustrates that the device is placed so that the arms are slightly below the leaflets which are held in their open position by the stent, while (B) illustrates that when the device is released the contraction of the left ventricle loads the valve pushing the anchoring arms up behind the leaflets and captures them atraumatically against the stent. As will be appreciated from FIG. 3, as the LV exerts pressure on the valve mechanism, the arms are pushed up behind the anterior and posterior leaflets. This mechanism allows the valve leaflets to be gently trapped between the stent body and the arms. In the region of the commissures where leaflet tissue can be sparse, the arms tend to grasp chordae up near the annulus. This mechanism is remarkably strong yet completely atraumatic.

Additionally, the device of FIG. 1 is designed for antegrade delivery. This delivery strategy avoids the problems some of the other groups have reported with retrograde approaches—specifically having the expansion and positioning of their devices impeded by obstruction of the chordae. The device of FIG. 1 also makes the minimally invasive surgical procedure safer. A small incision in the atrium is safer and easier to make than an incision into the apex of the LV (retrograde placement).

The device shown in FIG. 1 has been placed in 8 sheep as a sutureless mitral valve using standard open heart surgical technique. The device is introduced into the mitral valve annulus using a 30 french (30 F) introducer. Placement takes literally seconds and cross clamp times have been less than 10 minutes. In five animals, the device was found to function well with secure anchoring and no perivalvular leak or LVOT obstruction. For these experiments, animals were euthanized after 12 hours to assess the anchoring and sealing mechanism directly. The device functioned well in three animals for a week after which the animal was euthanized for direct device evaluation (FIGS. 1C and 1D).

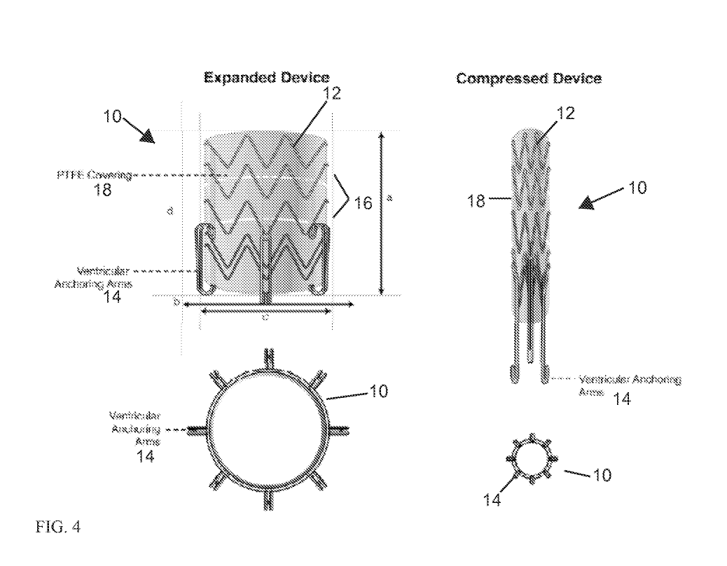

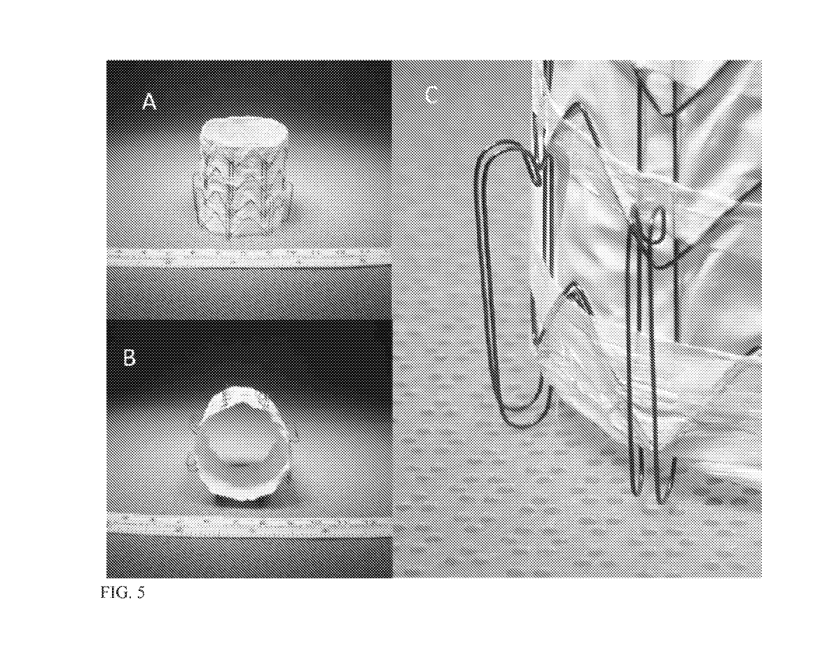

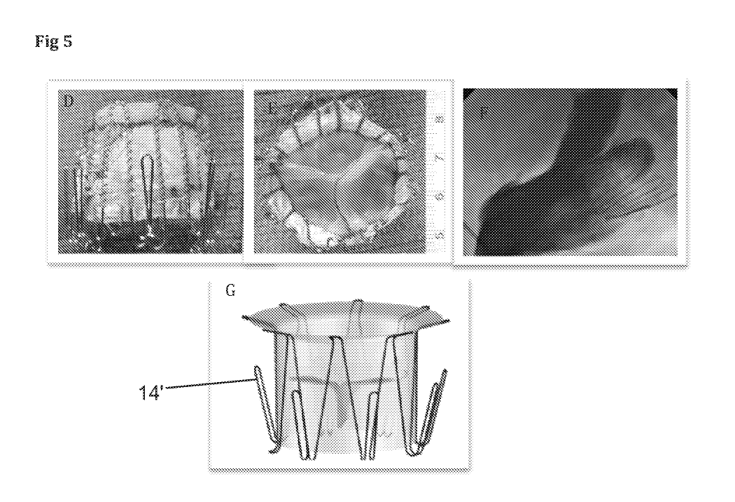

In order to enhance foldability and perivalvular seal, the inventors have developed the embodiments shown in FIGS. 4 and 5 in accordance with the present invention. In these designs, nitinol has been minimized to facilitate compression during insertion with the majority of the stent being created from thin PTFE. FIG. 4 illustrates a first embodiment of a PTFE-nitinol wire valve prosthetic device in accordance with the invention. The device is shown on the left in expanded position and on the right in its folded transcatheter delivery position. As illustrated in FIG. 4, the valve prosthesis includes a partially self-expanding stent 10 having a nitinol wire framework 12 defining outer and interior surfaces, anchoring arms 14 and a middle region 16. The stent 10 has an unexpanded and an expanded state, and the anchoring arms 14 have hooks that hook around the leaflets when seated. The middle region 16 is covered by an elastic fabric/cloth 18 that is wrapped around the wire framework 12 that is useful to form a seal when seated. The prosthesis includes a valve (not shown) having at least one leaflet fixedly attached to the interior surface of the stent 10. In slaughterhouse heart testing, this embodiment has been found to be remarkably softer and more adherent to the mitral valve annulus than the all-nitinol wire weave device of FIG. 1. Despite having less than ¼ the number of arms (8 vs. 32), it anchors as effectively as the all-nitinol device did in vitro. Such a significant reduction in the number of arms (e.g., 4-20 arms instead of the 25+ arms in the embodiment of FIG. 1) will significantly lower the device's profile and enhance transcatheter deliverability. Also, the higher “pitch” of the wire framework 12 in this embodiment (e.g., 4-10 transversals of the circumference of the stent 10) compared to the device of FIG. 1 results in the use of even less wire and hence a further reduced device profile. Such design features further facilitate placement of the device in “over-sized mitral annuli (>4 cm).

FIGS. 5A-5C illustrate an embodiment of the device of FIG. 4 where (A) is a side view, (B) is a view of the device from ventricular to atrial end, and (C) is a close up view of the anchoring arm design. FIGS. 5D-5E show side and end face views of an alternative embodiment of the device of FIGS. 5A-5C in which the wire framework has a higher amplitude extending the length of the stent and a lower frequency (fewer traversals of the circumference of the stent) than that shown in FIGS. 5A and 5B. Instead of multiple wire zigs, as shown in FIGS. 5A and 5B, the supporting framework includes a single stainless steel (or nitinol) wire arranged in a ring of high amplitude running the length of the stent 10 and varying frequency (4-20) peaks, which form anchoring arms on the ventricular end in the device. The radial force in this configuration is maintained by varying amplitude, pitch and thickness of the wire used (0.005″-0.03″). FIG. 5F shows a radiographic view of the device pictured in 5D-5E implanted within the mitral annulus. FIG. 5G shows yet another embodiment of the device in which the atrial aspect of the device is flared circumferentially outward from the center, terminating in atrial arms 12′ that enhance device deliverability, anchoring, and seal.

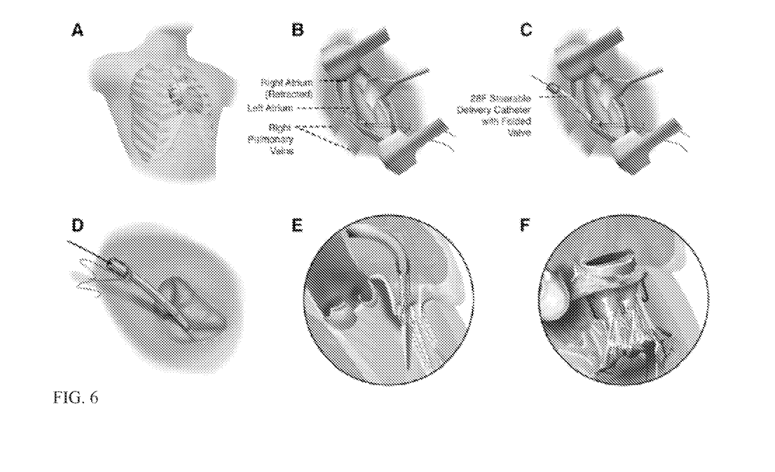

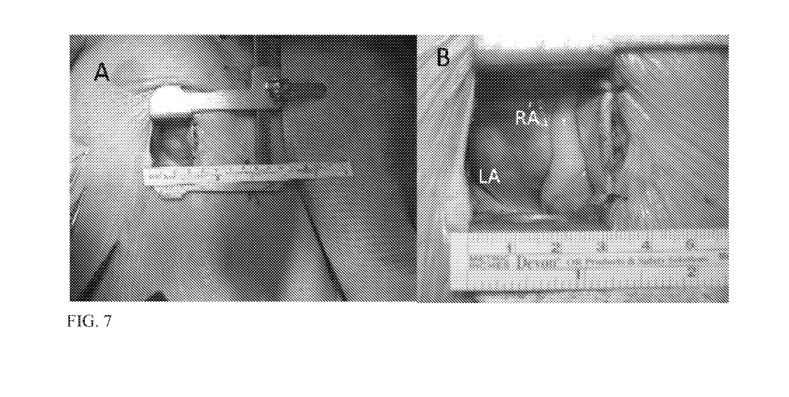

The devices of FIGS. 4 and 5 are designed to facilitate the replacement of the mitral valve via a small (3 cm or less) right thoracotomy, a purse string suture controlled left atrial access site and no need for CPB, as shown in FIG. 6. As shown in FIG. 6, a 3 cm incision is made in the 4^{th} anterior right intercostal space (A) and the right atrium is retracted (B). The device introducer is placed into the left atrium at (C), and the device is placed and secured in the mitral valve annulus as shown at (D), (E), and (F). Currently such small incisions are used routinely by some surgeons to repair mitral valves using CPB and thoracoscopic surgical techniques such an incision as shown in FIG. 7. As shown in FIG. 7 at (A), the patient is in a partial left lateral decubitus position and a 3 cm incision has been made in the right anterior 4^{th} intercostals space. The pericardium has been incised and retracted to expose the interatrial groove. (B) illustrates a close-up view of the exposed heart, where LA is the left atrium and RA is the right atrium. Such an approach is designed to eliminate the morbidity of both a large incision and CPB for patients requiring valve replacement.

Also, the device of FIGS. 4 and 5 is delivered via a transvenous/transatrial septal delivery technique for mitral valve replacement. Within the heart the delivery angles are very similar between the minimally invasive surgical (MIS) approach and the percutaneous trans-septal approach. This facilitates the easy incorporation of the MIS technology into the transvenous delivery catheter design. Additionally, the transvenous approach allows for the safer use of larger delivery catheters and reduces the risk of vascular complication which has plagued the transcatheter aortic valves currently in use clinically which require placement via the femoral or iliac arteries.

### Optimization of the Anchoring Arm Design

In extensive animal work with the nitinol wire weave design of prior art FIG. 1, the inventors have found that optimal anchoring and sealing occurs when the device is seated completely within the cone-shaped space created by the annulus and open leaflets (leaflet cone) as shown in FIG. 2. Real-time 3-D echocardiography (rt-3DE) techniques were used to non-invasively assess leaflet and annular geometry as well as physiology. These rt-3DE techniques have been applied in conjunction with the Philips IE33 platform to precisely image the mitral annular leaflet cone in large healthy sheep (80 kg) used to test the devices. The inventors have found that when the devices of FIG. 1 are sized with a diameter of 35 mm and a length of 30 mm they fit snuggly and completely within the leaflet cone.

The successful nitinol weave prototypes for the device of FIG. 1 have had 25 arms whose lengths were 75% of the stent body length. Based on extensive slaughterhouse heart testing with the PTFE-nitinol design of FIG. 1; however, the inventors believe that both the number of arms and their lengths can be reduced significantly. While the inventors have found slaughterhouse heart testing to be predictive of in vivo anchoring arm function, it is not precise enough to base final design criteria on for several reasons: first, the arm mechanism relies on LV loading for orientation; second, while fewer and shorter arms enhance foldability, arm length also influences positioning within the leaflet cone. If the arms are too long, the device can be held partially beneath the leaflets, which promotes LVOT obstruction and an ineffective seal. On the other hand, if the arms are too short, anchoring strength is diminished. Due to these complex interactions, iterative in vivo testing was necessary to define the optimal length and number of anchoring arms for the PTFE-nitinol design.

The inventors note that there are varying combinations of arm number and length that may work optimally. Because arm number influences folding and anchoring most significantly, the arm number is optimized first by constructing PTFE-nitinol prototypes with dimensions specified above and a varying number of arms (20, 16, 12 and 8) of the same length (0.75 arm length to stent length ratio). Each device was fitted with a custom designed trileaflet pericardial valve and optionally included a polyester skirt. The leaflets were designed for optimal opening and closing during the cardiac cycle and were cut from bovine pericardium with a thickness ranging from 0.23 mm to 0.28 mm. The skirt provided attachment for the leaflets and acted as an interface between the leaflets and the stent. The entire assembly was sutured together using a size 6-0 Tevdek II white braided PTFE impregnated polyester fiber suture.

Human-sized sheep (80 kg) were anesthetized and a left anterior thoracotomy performed. The pericardium was opened to expose the heart and an epicardial rt-3DE evaluation of the mitral valve was performed. The animal was then placed on CPB using standard cannulation techniques. Using standard open heart techniques, the mitral valve was exposed through a left atriotomy. A custom made applicator was then used to place the devices of FIGS. 4 and 5 through the mitral annulus into the LV and then pulled back partially into the leaflet cone as it was released. The atriotomy was then closed. The aortic cross clamp was removed and the animal weaned from CPB. After placement, the device assessed by rt-3DE for stability and perivalvular seal. If function was satisfactory (proper orientation, valve function, and seal) the animal was allowed to survive for 1 month and the valve reassessed by rt-3DE. If the device was not functioning appropriately, the animal was euthanized and the heart removed for direct visual assessment of valve malposition/malfunction. Each arm number design was tested in 5 animals.

Arm length was optimized by using the successful device with the fewest arms (as determined above) with varying arm lengths (0.6, 0.4, 0.3, 0.2 ASR). Each device was fitted with a pericardial valve as previously described. Each arm length was evaluated in 5 animals. The same iterative evaluation, imaging techniques and surgical procedures were used as in the above example. The 0.6 ASR prototypes were assessed first with sequentially shorter arms being tested subsequently. The successful prototype was that which functioned adequately with the shortest and fewest arms.

It is the inventors' belief that the added flexibility of the PTFE design not only makes it more foldable for delivery purposes but its flexibility has been found to make it more adherent to the leaflet cone. This added adherence makes it more efficient in perivalvular sealing with fewer and shorter arms than used in the nitinol wire weave designs such as in FIG. 1. In the exemplary embodiments of FIGS. 4 and 5, the PTFE device functions effectively with no more than 12 arms that are 40% of the length of the stent body. Based on this arm geometry and the current leaflet design, the inventors have found that with routinely available folding techniques such a device can be delivered through a 22-24 F introducer. Also, the arm-leaflet interaction is believed to be an important contributor to the seal in addition to being part of the fixation system.

### Optimization of the Delivery System Design

Two types of steerable, coaxial, delivery, deployment and retrieval systems may be used to deliver the device to the mitral valve position. The first system is designed to allow placement of the valve through a small thoracotomy and purse string controlled atriotomy (i.e., a minimally invasive surgical procedure: MIS). The second system allows for valve placement via a trans-femoral vein/trans-atrial septum approach to the mitral valve. Both systems are tailored to accommodate the arm design of the TMVR device optimized above. For each system, the length, width, radius of curvature, release mechanism, and docking station characteristics are defined.

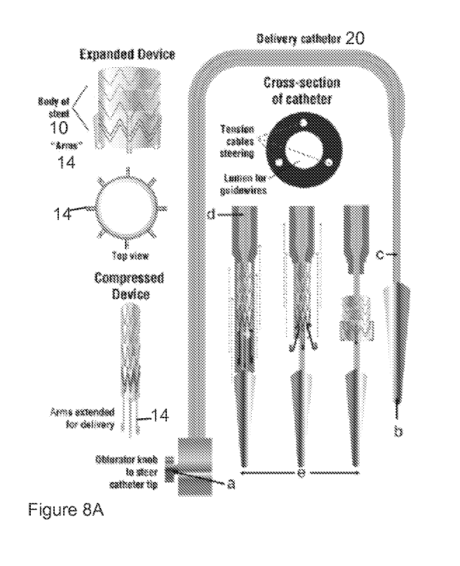

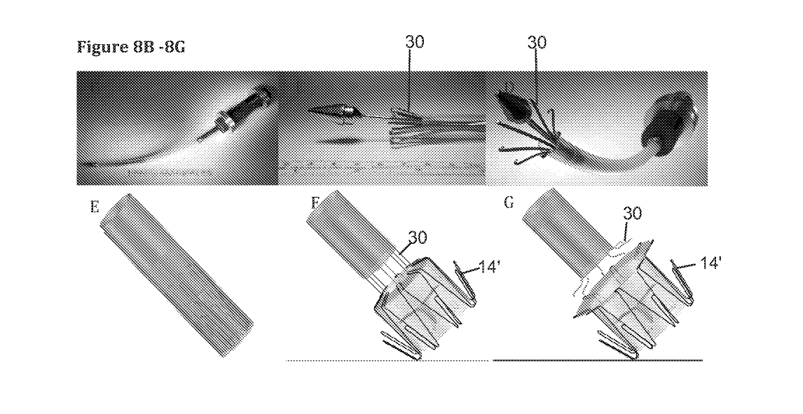

The essentials of a first embodiment of a delivery system design are shown in FIG. 8A. As illustrated, tension wires that run the length of the catheter 20 are controlled by an obdurator control knob (a). The leading tip (b) is tapered for easy atraumatic insertion. (c) is the device docking position, while (d) and (e) illustrate the dual compression sleeve mechanism. Withdrawing the outer sleeve allows the arms 14 to position themselves while withdrawal of the inner sleeve allows expansion of the stent body.

FIGS. 8B-8D illustrate an alternative embodiment of a delivery system in which the peaks of the device frame at the atrial (proximal) end of the device are grabbed by a claw mechanism 30 that collapses the device centrally to reduce the profile for delivery via catheter. This claw mechanism 30 facilitates robust control of the proximal end of the device during deployment. Proximal control during delivery may also be enhanced using a suture noose (single or multiple) or coil (screw) mechanism (not shown). FIGS. 8E-8G illustrate schematic representations of the step-wise expansion and eventual release of the device of FIG. 5G from the claw mechanism 30 of the embodiment of FIGS. 8B-8D.

### Mini Thoracotomy Delivery

Using standard surgical techniques, a sterile left 3 cm anterior thoracotomy is performed and the left atrium exposed (unlike the human the left atrium is more easily reached via a small left thoracotomy rather than a right in a sheep). An atrial purse string is placed, through which an angiographic catheter is introduced across the MV annulus into the LV. A stiff 0.035″ guidewire is introduced and looped in the LV apex. The TMVR device is loaded into the delivery catheter and then introduced through the purse string, over the wire, into the atrial chamber, and across the MV annulus.

Given the dynamic nature of the MV annulus in the beating heart, visualization of the annular plane, leaflets, and submitral apparatus are essential for accurate transcatheter deployment of the TMVR device. A combination of angiography, and intracardiac echocardiography (ICE), and rt-3DE is used for localization of the important mitral valve components. Once appropriate positioning is confirmed via these imaging modalities, the TMVR device is deployed. Follow up rt-3DE and angiography are used to assess TMVR device position, function, and stability. The delivery system is withdrawn once stable position is established. The atrial purse string and thoracotomy are repaired in the standard fashion.

### Percutaneous Delivery

The general folding, imaging and delivery strategy is the same as developed for the MIS procedure. Catheter steerability is needed for percutaneous placement. As shown in FIG. 8A, a 3 cable control mechanism may be used in an exemplary embodiment. Alternatively, as shown in FIGS. 8B-G, a claw mechanism may be used for percutaneous placement. In either case, the catheter has several important components that allows for transport through the vasculature and controlled deployment and release of the TMVR device:
a. The catheter has tension cables running longitudinally along the length of the device, allowing for deflection of the catheter tip or steerability. This is controlled by an obdurator knob located proximally on the catheter;
b. The leading tip of the catheter is tapered, to allow for easy insertion into the femoral vein and atraumatic advancement though the vasculature;
c. The TMVR device is compressed and loaded into a dock at the distal aspect of the catheter, located just proximal to the tapered leading tip;
d. The TMVR device is held securely within the dock by 2 compression sleeves arranged coaxially; and
e. For deployment of the TMVR device, the compression sleeves are withdrawn proximally in a sequential manner, allowing the self-expanding TMVR device to expand. Retraction of the outer sleeve allows the ventricular arms of the device to swing back towards the body of the TMVR device and, in the process, to begin to insinuate themselves around leaflet and chordal tissue. Retraction of the inner sleeve allows the body of the TMVR device to expand and in doing so to capture the leaflets between stent body and anchoring arms.

Not shown in FIG. 8, but an important element in the delivery system, is a retrieval cord, which is attached to the proximal aspect of the TMVR device during loading into the dock. This cord extends through the body of the catheter and out a port in the proximal end. It prevents premature release and allows device retrieval if placement is suboptimal.

Due to the longer route to the left atrium, there is some necessary optimization of catheter length, width, and radius of curvature. However, the release mechanism and docking station characteristics are the same as for the MIS delivery device. As in the experiments described above, appropriate visualization is critical to successful TMVR deployment, and so an imaging protocol is used.

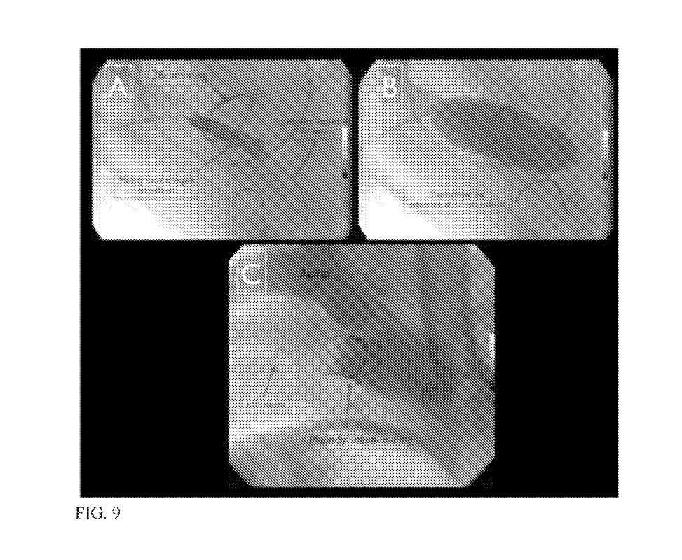

The inventors have previously demonstrated the feasibility of mitral valve replacement in the beating heart using the systemic venous circulation and transatrial septal puncture. This work was done in animals with pre-existing annuloplasty rings—the so-called valved stent-in-ring (VIR) procedure as shown in FIG. 9. In this embodiment, a failed mitral valve repair is treated using an annuloplasty ring. This embodiment makes stent replacement of the valve much easier. As illustrated in FIG. 9 at (A), the valved stent is crimped on the delivery balloon and advanced over the guide wire from the femoral vein, across the atrial septum and positioned centrally in the annuloplasty ring. (B) shows deployment of the valved stent via balloon inflation, while (C) shows a follow-up left ventriculogram. There is no mitral regurgitation and no left ventricular outflow tract obstruction. An atrial closure device is used to close the small atrial septal defect.

In the embodiment of FIG. 9, the anchoring arms are not needed to anchor the valve prosthesis. Access to the femoral vein is obtained via surgical cutdown. Using ICE guidance, an atrial transeptal puncture is performed and an atrial septal defect (ASD) is created via balloon dilation. A super-stiff 0.035″ preformed guidewire is looped in the LV apex, forming a rail from the iliac vein, across the ASD and MV into the LV. Next, the TMVR device is loaded into the delivery catheter, and the catheter is introduced into the femoral vein over the wire and advanced into position at the mitral annulus as shown in FIG. 9. Based on the compressed profile of the TMVR, the delivery catheter outer diameter may be, for example, approximately 24 F.

Once the proper device position is confirmed using ICE, rt-3DE, and/or angiography, the TMVR device is deployed, released, and assessed for location and stability. In particular, the stent of the TMVR device in this embodiment is expanded until it is held in place by radial pressure against said annuloplasty ring. In exemplary embodiments, the annuloplasty ring and/or the stent may have a magnet and/or a detent incorporated therein such that the expanded stent does not move relative to the annuloplasty ring due to magnetic force retention and/or interaction with the detent. The delivery system is withdrawn once stable position is established. The ASD is closed via standard transcatheter techniques.

### Long Term TMVR in an Ovine Model of IMR

For testing of the devices described herein, the inventors have developed and extensively studied a sheep model of IMR which mimics the human disease very precisely. The model is produced by ligating the second and third branches of the circumflex artery. Twenty to 25 percent of the posterior basal LV myocardium is reliably infarcted and 3 to 4+MR develops over 8 weeks. The inventors have quantitatively characterized this IMR model using rt-3DE and analysis software. Using an extensive library of quantitative rt-3DE images, the size and the geometry of the leaflet cone in sheep with IMR is assessed. This data is then used to optimize the size of the device for IMR sheep. These prototypes are then placed using both the MIS and TMVR delivery systems described above.

Those skilled in the art will also appreciate that the invention may be applied to other applications and may be modified without departing from the scope of the invention. For example, those skilled in the art will appreciate that the devices and techniques of the invention may be used to replace the tricuspid valve as well as the mitral valve. Also, those skilled in the art will appreciate that the device may be made of stainless steel of varying thickness instead of nitinol. Accordingly, the scope of the invention is not intended to be limited to the exemplary embodiments described above, but only by the appended claims.

What is claimed:

1. A valve prosthesis comprising:
an at least partially self-expanding stent comprising a wire framework defining outer and interior surfaces and an anchoring arm, said stent having an unexpanded and an expanded state, and said anchoring arm having an elbow region and a hook that clamps around mitral tissue of the patient when seated;
an elastic fabric/cloth that is wrapped circumferentially around the wire framework; and
a valve comprising at least one leaflet fixedly attached to the interior surface of said stent.

2. The valve prosthesis of claim 1, wherein the elastic fabric/cloth comprises a PTFE material.

3. The valve prosthesis of claim 1, wherein said stent comprises between 4 and 20 anchoring arms.

4. The valve prosthesis of claim 3, wherein said anchoring arms have lengths that are 40% of a length of the stent.

5. The valve prosthesis of claim 1, wherein said anchoring arms are flared circumferentially outward.

6. The valve prosthesis of claim 1, wherein said wire framework traverses the circumference of the stent with a pitch that extends a portion of the length of the stent or the entire length of the stent 4-10 times.

7. A valve prosthesis comprising:
an at least partially self-expanding stent comprising a wire framework defining outer and interior surfaces, said stent having an unexpanded and an expanded state;
an elastic fabric/cloth that is wrapped circumferentially around the wire framework;
a valve comprising at least one leaflet fixedly attached to the interior surface of said stent; and
an annuloplasty ring into which said stent is inserted prior to expansion,
wherein said stent is adapted to be expanded to be held in place by radial pressure against said annuloplasty ring.

8. The valve prosthesis of claim 7, wherein the annuloplasty ring and/or the stent has a magnet incorporated therein such that the expanded stent does not move relative to the annuloplasty ring due to magnetic force retention.

9. The valve prosthesis of claim 7, wherein the annuloplasty ring and/or the stent has a detent incorporated therein such that the expanded stent does not move relative to the annuloplasty ring due to interaction with the detent.
